(19) United States
(12) Patent Application Publication
(10) Pub. No.: US 2004/0242907 A1
(43) Pub. Date: Dec. 2, 2004

(54) Methods of synthesizing flavonoids and chalcones

(75) Inventors: Jiajiu Shaw, Ann Arbor, MI (US); An-Rong Lee, Taipei (TW); Wen-Hsin Huang, Taipei (TW)

Correspondence Address: BRINKS HOFER GILSON & LIONE, P.O. BOX 10395, CHICAGO, IL, 60610, US

(73) Assignee: Unitech Pharmaceuticals, Inc.

(21) Appl. No.: 10/856,033

(22) Filed: May 28, 2004

Related U.S. Application Data
(60) Provisional application No. 60/474,371, filed on May 30, 2003.

(51) Int. Cl. C07D 311/04

(52) U.S. Cl. 549/403

(57) ABSTRACT

Simple and efficient total syntheses of flavonoids including baicalein, oroxylin A and wogonin are described herein. Simultaneous syntheses of oroxylin A and wogonin are also described.

### RELATED APPLICATIONS

This application claims the benefit of U.S. Provisional Application No. 60/474,371, filed May 30, 2003, the entire contents of which are incorporated herein by reference, except that in the event of any inconsistent disclosure or definition from the present application, the disclosure or definition herein shall be deemed to prevail.

### FIELD OF THE INVENTION

The present invention relates to simple and efficient synthetic methods for the syntheses of flavonoids and, in particular, for the synthesis of baicalein, oroxylin A, and wogonin.

### BACKGROUND

Baicalein, oroxylin A, and wogonin are the three major flavonoids of Scutellaria baicalensis Georgi, a traditional Chinese herb used since ancient times, which possesses a very broad spectrum of biological activities, notably anti-oxidant.

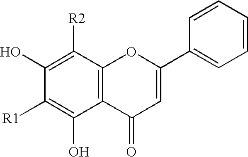

| | R1 | R2 |
|---|---|---|
| baicalein | OH | H |
| oroxylin A | OCH₃ | H |
| wogonin | H | OCH₃ |

To the best of our knowledge, there are currently no appropriate approaches available for the facile synthesis of these three flavonoids. In view of their unique pharmacological properties, development of a pertinent route for the efficient preparation of the three specific flavonoids, baicalein, oroxylin A, and wogonin, would be highly desirable.

In general, procedures for laboratory synthesis of flavonoids are still based on the approaches originally developed by Robinson, on synthesis via chalcones developed by Iinuma, and/or synthesis via intramolecular Wittig reactions. At the outset, we followed the reported methods for synthesizing flavonoids to make the three specific flavonoids, baicalein, oroxylin A and wogonin. However, we found that all of the reported methods proved unsatisfactory, either in requiring too many steps thereby resulting in low overall yields, or by involving irreproducible workups thereby creating considerable challenges to reproducibility. Attempts to synthesize baicalein from trimethoxyphenol by either the modified conventional or Baker-Venkataraman approach proved to be impractical (e.g., less than 10% yield), while attempts to utilize the Wittig strategy failed completely.

### SUMMARY

The scope of the present invention is defined solely by the appended claims, and is not affected to any degree by the statements within this summary.

A first method of synthesizing a flavonoid embodying features of the present invention includes: (a) reacting a chalcone having a structure (1)

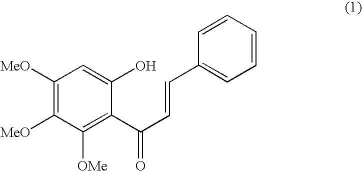

under demethylating conditions to form an at least partially demethylated chalcone; and (b) reacting the at least partially demethylated chalcone under oxidative conditions to form a flavonoid selected from the group consisting of oroxylin A, wogonin, and a combination thereof.

A second method of synthesizing a flavonoid embodying features of the present invention includes reacting a flavone having a structure (2)

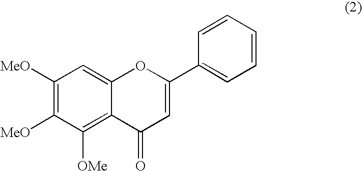

under demethylating conditions to form a flavonoid selected from the group consisting of oroxylin A, baicalein, and a combination thereof.

A method of synthesizing a chalcone having a structure (1):

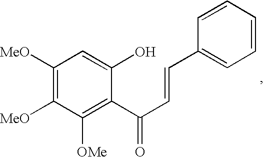

includes acylating 3,4,5-trimethoxyphenol either directly or indirectly.

### BRIEF DESCRIPTION OF THE DRAWING

FIG. 1 shows synthetic schemes in accordance with the present invention for synthesizing baicalein, oroxylin A, and wogonin.

### DETAILED DESCRIPTION

A unique and efficient strategy for synthesizing baicalein, oroxylin A, and wogonin has been discovered, wherein demethylation is performed at the last stage. Results indicate that this strategy works surprisingly well. The present inventors have described features of the present invention in Chem. Pharm. Bull. 2003, 51, No. 3, pp. 339-340, the entire contents of which are incorporated herein by reference, except that in the event of any inconsistent disclosure or definition from the present application, the disclosure or definition herein shall be deemed to prevail.

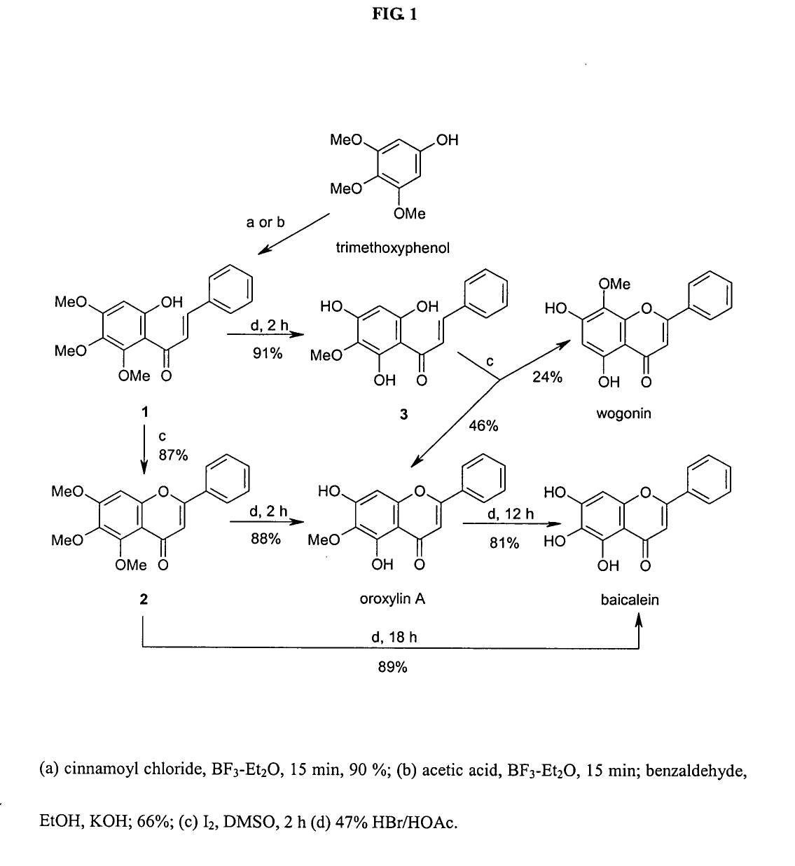

Approaches in accordance with the present invention for synthesizing baicalein, oroxylin A, and wogonin are shown in FIG. 1, and involve preparation of flavone 2 and chalcone 3 as penultimate targets derived from chalcone 1. Chalcone 1 is readily prepared by treatment of readily accessible 3,4,5-trimethoxyphenol with excessive acetic acid in the presence of BF₃-Et₂O, followed by a Claisen-Schmidt condensation with equimolar benzaldehyde, preferably catalyzed by KOH, in 66% overall yield. Alternatively, a better yield (90%) was achieved by direct acylation of 3,4,5-trimethoxyphenol with equimolar cinnamoyl chloride, also in the presence of BF₃-Et₂O.

One of the most common methods for the preparation of flavonoids, such as flavone 2, involves an intramolecular oxidative cyclization of a chalcone, such as chalcone 1. However, formation of the prerequisite flavone 2 triggered by SeO₂/EtOH or Pd(OAc)₂/AcCN consistently led to extremely low yields (below 10%). This difficulty of cyclization is believed to be due to the phenyl ring bearing polyphenols (more than 3 OH's). Accordingly, non-metal oxidants were investigated. Among them, I₂/DMSO proved to be the most promising and, surprisingly, the reaction proceeded smoothly and provided flavone 2 in a much superior yield (87%). Surprisingly and unexpectedly, attempted demethylation of flavone 2 in a solution of 47% HBr/AcOH (1:2) at reflux for 2 h to give baicalein instead gave, after isolation, an unexpected yet desirable product exclusively: oroxylin A (88%). Further reaction under the same conditions over 12 h yielded baicalein (81%). Alternatively, a straight 18-h hydrolysis of flavone 2 employing the same methodology also afforded baicalein in excellent yield (89%).

In similar fashion, demethylation of chalcone 1 in a solution of 47% HBr/HOAc (1:2) at reflux for 2 h gave chalcone 3 (91%), which was susceptible to oxidation with I₂/DMSO to give a mixture of oroxylin A (46%) and wogonin (24%), which are readily separated by flash chromatography.

A number of derivatives of chalcone 1 and derivative of baicalein (e.g., 4g and 9i) may also be made by methods in accordance with the present invention.

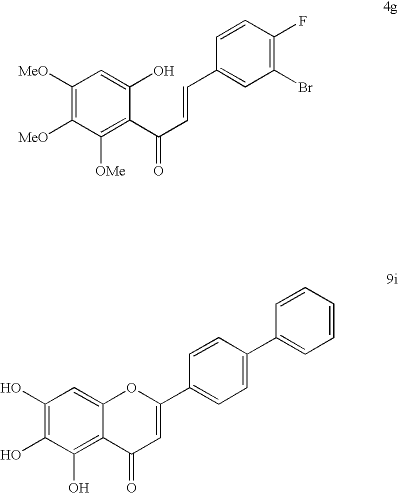

The following representative examples and procedures are provided solely by way of illustration, and are not intended to limit the scope of the appended claims or their equivalents.

### EXAMPLES

Melting points were determined with a Buchi-530 melting point apparatus (uncorrected). IR spectra were recorded on a Perkin-Elmer FT-IR 1600 series FT-IR spectrophotometer. ¹H NMR spectra were determined on a Varian Gemini-300 NMR. Mass spectra were recorded on a Finnigan MAT TSQ-46 or Finnigan MAT TSQ-700. UV spectra were recorded on a Shimadzu UV-160A.

### Example 1

1-(6-Hydroxy-2,3 4-Trimethoxyphenyl)-3-Phenyl-Propenone (Chalcone 1)

A mixture of 3,4,5-trimethoxyphenol (3.7 g, 20 mmol) and cinnamoyl chloride (3.7 g, 22 mmol) was dissolved in BF₃-Et₂O complex (20 ml) and heated to reflux for 15 min, monitored by TLC (hexane:EtOAc=3:1), and then quenched with an excess of water. Filtration and recrystallization from hexane:EtOAc (3:1) procured chalcone 1 (5.6 g, 90%). Alternatively, 3,4,5-trimethoxyphenol (3.7 g, 20 mmol) was acylated with acetic acid (1.7 ml, 30 mmol) in BF₃-Et₂O complex (20 ml) under reflux for 15 min to give a ketone intermediate (3.8 g, 83%), which was reacted without further purification with benzaldehyde (1.8 ml, 17 mmol) in ethanol (20 ml). The mixture was added to 50% KOH (7.6 g) below 15° C. and then stirred at room temperature, preferably for 8 h under nitrogen. The reaction was quenched with an excess of ice water. The mixture was acidified with 6-N HCl and then partitioned in EtOAc and water. The organic layer was washed with brine and dried with anhydrous Na₂SO₄. Filtration and removal of the solvent afforded after recrystallization with hexane:EtOAc (3:1) 4.3 g (80%) of 1: mp 98-100° C. ¹H-NMR (CDCl₃) δ: 3.82 (3 H, s), 3.96 (3 H, s), 4.03 (3 H, s), 6.34 (1 H, s), 7.45-7.48 (3 H, m), 7.67 (2 H, d, J=9.3 Hz), 8.06 (2 H, d, J=15.5 Hz), 8.33 (2 H, d, J=15.5 Hz). IR (KBr) cm⁻¹: 3419, 1608. MS m/z: 315 (MH⁺).

### Example 2

5,6,7-Trimethoxyflavone (Flavone 2)

A mixture of chalcone 1 (7.2 g, 23 mmol) and iodine (200 mg) in DMSO (25 ml) was refluxed for 2 h, and then carefully poured onto crushed ice (200 g). The precipitate was filtered and washed with 20% Na₂SO₃. Purification by flash column chromatography (SiO₂, hexane:EtOAc=3:1) yielded 6.3 g (87%) of flavone 2 and 0.8 g (2.5%) of recovered chalcone 1: mp 146-147° C. (lit. 164-165° C.). ¹H-NMR (DMSO-d₆) δ: 3.93 (3 H, s), 3.97 (3 H, s), 3.99 (3 H, s), 6.72 (1 H, s), 6.83 (1 H, s), 7.50 (3 H, m), 7.88 (2 H, d, J=8.7 Hz). IR (KBr) cm⁻¹: 1633. MS m/z: 313 (MH⁺).

### Example 3

Oroxylin A

A solution of flavone 2 (0.20 g, 0.64 mmol) in 47% HBr (5 ml) and glacial acetic acid (10 ml) was refluxed for 2 h, and then carefully poured onto crushed ice (200 g). The resulting yellow precipitate was filtered and collected. Recrystallization from ethanol afforded 160 mg (88%) of oroxylin A: mp 203-204° C. ¹H-NMR (DMSO-d₆) δ: 3.91 (3 H, s), 6.94 (1 H, s), 6.98 (1 H, s), 7.59 (3 H, m), 8.10 (2 H, d, J=6.3 Hz), 8.77 (1 H, s), 12.49 (1 H, s). IR (KBr) cm⁻¹: 3435, 1667. UV λₘₐₓ (EtOH) nm (log ε): 322 (4.12), 278 (4.35), 216 (4.42). MS m/z: 285 (MH⁺).

### Example 4

Baicalein

Baicalein was prepared by the modified procedure outlined above either from oroxylin A (reflux, 12 h) or from flavone 2 (reflux, 18 h) in 81% and 89% yields, respectively: mp 258-260° C. (lit. 263-264° C.). R_{f} (CH₂Cl₂:EtOAc=5:1) 0.4. ¹H-NMR (DMSO-d₆) δ: 6.61 (1 H, s), 6.92 (1 H, s), 7.56 (3 H, m), 8.05 (2 H, d, J=8.1 Hz), 8.81 (1 H, s), 10.57 (1 H, s), 12.65 (1 H, s). IR (KBr) cm⁻¹: 3411, 1654. UV λₘₐₓ (EtOH) nm (log ε): 326 (4.17), 276 (4.42), 215 (4.49). MS m/z: 270 (M⁺).

### Example 5

Wogonin

Pure chalcone 3 (0.52 g, 1.8 mmol), prepared by the procedure outlined above (reflux, 2 h) from chalcone 1 (0.62 g, 2.0 mmol) in 91% yield, was subjected to oxidative cyclization as previously described. Purification by flash chromatography (silica gel, CH₂Cl₂→hexane:EtOAc (3:1)→CH₂Cl₂:EtOAc (5:1)) and then recrystallization from ethanol gave oroxylin A (238 mg, 46%) and wogonin (124 mg, 24%), respectively. Chalcone 3: mp 121-122° C. R_{f} (CH₂Cl₂:EtOAc=5:1) 0.69. ¹H-NMR (DMSO-d₆) δ: 2.78 (1 H, d, J=13.4 Hz), 3.82 (3 H, s), 5.56 (1 H, d, J=13.4 Hz), 6.27 (1 H, s), 7.40-7.54 (5 H, m), 8.21 (1 H, s), 11.72 (1 H, s). IR (KBr) cm⁻¹: 3445, 1666. FAB-MS m/z: 287 (MH⁺); Wogonin: mp 198-199° C. R_{f} (hexane:EtOAc=3:1) 0.41, (CH₂Cl₂:EtOAc=5:1) 0.68. ¹H-NMR (DMSO-d₆) δ: 3.81 (3 H, s), 6.30 (1 H, s), 7.00 (1 H, s), 7.37 (1 H, s), 7.64 (3 H, m), 8.10 (2 H, d, J=6.3 Hz), 12.51 (1 H, s). IR (KBr) cm⁻¹: 3445, 1667. UV λₘₐₓ (EtOH) nm (log ε): 321 (4.15), 276 (4.36), 216 (4.44). FAB-MS m/z: 285 (MH⁺).

In accordance with the present invention, an extremely efficient route for the preparation of baicalein, oroxylin A, and wogonin has been discovered. To the best of our knowledge, for the total synthesis of these three pharmacologically diversified flavonoids, the approach described herein is the only practical path beginning from a common starting material, using affordable reagents, and proceeding under mild conditions which, therefore, is suitable for large-scale pilot-plant synthesis. The synthetic scheme in accordance with the present invention is generally represented in FIG. 1.

The foregoing detailed description and examples have been provided by way of explanation and illustration, and are not intended to limit the scope of the appended claims. Many variations in the presently preferred embodiments illustrated herein will be obvious to one of ordinary skill in the art, and remain within the scope of the appended claims and their equivalents.

1. A method of synthesizing a flavonoid, comprising:
reacting a chalcone having a structure (1) 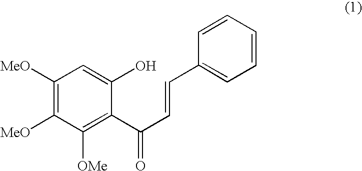
under demethylating conditions to form an at least partially demethylated chalcone; and
reacting the at least partially demethylated chalcone under oxidative conditions to form a flavonoid selected from the group consisting of oroxylin A, wogonin, and a combination thereof.

2. The method of claim 1 wherein the at least partially demethylated chalcone has a structure (3):

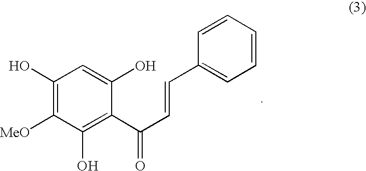

3. The method of claim 2 wherein the at least partially demethylated chalcone (3) is refluxed with I₂ in DMSO.

4. The method of claim 3 wherein the chalcone (1) is refluxed in 47% HBr/glacial acetic acid.

5. A method of synthesizing a flavonoid, comprising:
reacting a flavone having a structure (2) 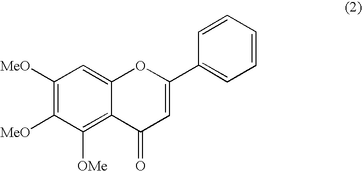
under demethylating conditions to form a flavonoid selected from the group consisting of oroxylin A, baicalein, and a combination thereof.

6. The method of claim 5 wherein the flavone (2) is prepared from a chalcone having a structure (1)

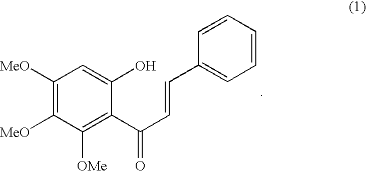

7. The method of claim 5, wherein the flavone (2) is refluxed in 47% HBr/HOAc for about two hours, and wherein the flavonoid comprises oroxylin A.

8. The method of claim 7 wherein the oroxylin A is formed in a yield of at least about 50 percent.

9. The method of claim 7 wherein the oroxylin A is formed in a yield of at least about 65 percent.

10. The method of claim 7 wherein the oroxylin A is formed in a yield of at least about 75 percent.

11. The method of claim 5, wherein the flavonoid comprises baicalein.

12. The method of claim 11 wherein the flavone (2) is reacted with 47% HBr/HOAc for a reaction time sufficient to allow substantially complete demethylation of flavone (2).

13. The method of claim 12 wherein the reaction time is longer than about two hours.

14. The method of claim 13 wherein the reaction time is longer than about six hours.

15. The method of claim 11, wherein the baicalein is formed in a yield of at least about 50 percent.

16. The method of claim 11, wherein the baicalein is formed in a yield of at least about 60 percent

17. The method of claim 11, wherein baicalein is formed in a yield of at least about 70 percent.

18. A method of synthesizing a chalcone having a structure (1): 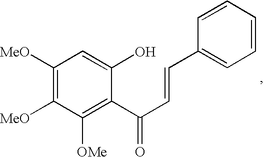
comprising acylating 3,4,5-trimethoxyphenol either directly or indirectly.

19. The method of claim 18 wherein the 3,4,5-trimethoxyphenol is reacted with cinnamoyl chloride in the presence of BF₃-Et₂O complex to form the chalcone (1).

20. The method of claim 18 wherein the 3,4,5-trimethoxyphenol is reacted with acetic acid in the presence of BF₃-Et₂O complex to form an intermediate, which is then is condensed with benzaldehyde in the presence of catalytic KOH to form the chalcone (1).
